SEQUENCE LISTING (6 sequences)

SEQ ID NO: 1 - protein, 7 aa, Homo sapiens
DMYSVGV

SEQ ID NO: 2 - DNA, 20 bp, Homo sapiens
agctccaccctgacgatcta

SEQ ID NO: 3 - DNA, 20 bp, Homo sapiens
atgtgcagggctgaagagat

SEQ ID NO: 4 - DNA, 20 bp, Homo sapiens
catgctgggggtcaaatagt

SEQ ID NO: 5 - DNA, 28 bp, Homo sapiens
ggaattcctatccacgctccgaacggca

SEQ ID NO: 6 - DNA, 30 bp, Homo sapiens
cccaagcttgcaacactggccataacatag

(19) United States
(12) Patent Application Publication
(10) Pub. No.: US 2003/0131363 A1
(43) Pub. Date: Jul. 10, 2003

(54) Heme-regulated eukaryotic initiation factor 2 alpha kinase knockout mice and methods for use

(75) Inventor: Jane-Jane Chen, Belmont, MA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Massachusetts Institute of Technology

(21) Appl. No.: 10/314,108

(22) Filed: Dec. 6, 2002

Related U.S. Application Data
(60) Provisional application No. 60/339,360, filed on Dec. 7, 2001.

(51) Int. Cl. A01K 67/027

(52) U.S. Cl. 800/9; 800/18

(57) ABSTRACT

The targeted disruption of the gene encoding the heme-regulated eIF2-kinase (HRI) in mice establishes that HRI, which is expressed predominantly in erythroid cells, regulates the synthesis of both alpha and beta globins in red blood cell precursors by inhibiting the general translation initiation factor eIF2. HRI is a physiological regulator of gene expression in the erythroid lineage and acts both as a sensor of heme and an effector controlling the initiation of translation. Accordingly regulation of HRI can be used to control proliferation of cells, especially cells of hematopoietic origin, using compositions and compounds identified using HRI deficient mice.

This application claims priority to U.S. Ser. No. 60/339,360 filed Dec. 7, 2001.

The United States government has rights in this invention by virtue of a grant from the National Institutes of Health, DK-16272 to J-J Chen.

### BACKGROUND OF THE INVENTION

The present invention is the use of a heme-regulated eukaryotic initiation factor 2Alpha kinase, and formulations thereof, and knockout animals for screening for compounds which alter the activity of the kinase.

Heme controls the synthesis of protein in reticulocytes. In heme-deficiency, there is diminished initiation of protein synthesis with disaggregation of polyribosomes. The principal mechanism of the inhibition of initiation of protein synthesis is the phosphorylation of the Alpha -subunit of the eukaryotic initiation factor 2, eIF-2Alpha. In addition to heme-deficiency, oxidized glutathione (GSSG) and low levels of double stranded RNA inhibit initiation by promoting phosphorylation of eIF-2Alpha.

The translation of mRNA in eukaryotic cells occurs in the cytoplasm. In the first step of initiation, free 80 S ribosomes are in equilibrium with their 40 S and 60 S subunits. In the presence of eIF-3, 40 S subunits bind the eIF-3 and eIF-4C to form a 43 S ribosomal complex; the binding of eIF-3 and eIF-4C to the 40 S subunit inhibits the joining of the 60 S subunit.

In the next step, eIF-2 binds GTP and the initiator tRNA, Met-tRNAf, in a ternary complex. The binding by eIF-2 is specific for both guanine nucleotides and for Met-tRNAf. The ternary complex now binds to the 43 S ribosomal complex to form the 43 S preinitiation complex. The 43 S preinitiation complex binds mRNA in an ATP-dependent reaction in which eIF-4A, eIF-4B, and eIF-4F form a complex with the mRNA. The product of the binding of mRNA to the 43 S structure is bound close to the ribosome and the AUG initiator codon is downstream from the cap structure.

The joining of the 48 S preinitiation complex and the 60 S subunit is catalyzed by eIF-5 which has a ribosome-dependent GTPase activity. The joining reaction is accompanied by the release of the initiation factors eIF-3 and eIF-4C, eIF-2 is translocated to 60 S subunit as a binary complex, eIF2-GDP. The product of the joining reaction is the 80 S initiation complex. Formation of the active 80 S initiation complex is the final step in initiation. The Met-tRNAf is positioned in the P (peptidyl) site on the ribosome for the start of polypeptide elongation.

The sequence of steps in the process of initiation affords several opportunities for regulation. These include the recycling of eIF-2 after its release as the eIF-2-GDP complex; the formation of the ternary complex; and the relative affinities of mRNAs for eIF-2 and for eIF-4A, -4B, and -4F in determining the relative rates of translation of the mRNAs.

Heme-deficiency inhibited initiation of protein synthesis is characterized by a brief period of control linear synthesis, followed by an abrupt decline in this rate and by disaggregation of polyribosomes, associated with a decrease in the formation of the eIF-2-Met-tRNAf-GTP ternary complex and the 40 S-eIF-2Met-tRNAf-GTP 43 S initiation complex. The fundamental mechanism for the inhibition is the activation of cAMP independent protein kinases that specifically phosphorylate the 38-kDa Alpha -subunit of eIF-2 (eIF-2 Alpha). Dephosphorylation of eIF-2 Alpha accompanies the recovery of protein synthesis upon addition of hemin to inhibited heme-deficient lysates.

Alpha

The heme-regulated eukaryotic initiation factor 2 Alpha (eIF-2) kinase, also called heme-regulated inhibitor (HRI), plays a major role in this Alpha process. HRI is a cAMP-independent p Alpha rotein kinase that specifically phosphorylates the subunit (eIF-2) of the eukaryotic initiation factor 2 (eIF-2). Phosphorylation of eIF-2 Alpha in reticulocyte lysates results in the binding and sequestration of reversing factor RF, also designated as guanine nucleotide exchange factor or eIF-2B, in a RF-eIF-2(Alpha P) complex; the unavailability of RF, which is required for the exchange of GTP for GDP in the recycling of eIF-2 and in the formation of the eIF-2-Met-tRNAf-GTP ternary complex, resulting in the cessation of the initiation of protein synthesis.

Investigations into mechanisms that control gene expression in mammals has focused largely on transcription, and, to a lesser degree, on post-transcriptional events related to the fate of specific mRNAs and their cellular localization. Nevertheless, increasing biochemical evidence points to another level of regulation during somatic and germ cell differentiation, namely translational control. Translational regulation can be achieved either by modifying the concentrations or activities of general translational factors (Mathews, et al. (2000). Origins and Principles of Translational Control, N. Sonenberg, Hershey,. W. B., and Mathews, M. B., ed. (Cold Spring Harbor: Cold Spring Harbor Laboratory Press)), or in an mRNA-specific manner, dependent upon the interaction of transacting factors with cis-acting sequences present on the mRNA (Eisenstein, R. S. (2000) Ann. Rev. Nutri. 20, 627-662; Rouault and Harford (2000). Translational control of ferritin synthesis In translational Control of Gene Expression. In Translational Control of Gene Expression, N. Sonenberg, Hershey, J. W. B., and Mathews, M. B., ed. (Cold Spring Harbor: Cold Spring Harbor Laboratory Press), pp. 655-670). Although direct evidence of its physiological role in the context of a whole animal was lacking, the phosphorylation of the alpha-subunit of translational initiation factor 2 (eIF2 Alpha) has been recognized as a key mechanism of global inhibition of translational initiation in vitro. eIF2 is a general translational initiation factor composed of three subunits: Alpha, Alpha and Alpha. eIF2 forms two types of binary complexes: active eIF2.GTP and inactive eIF2.GDP. eIF2.GTP distinguishes the initiator tRNA (Met-tRNAi), to which it binds, from elongator tRNAs by recognizing specific bases at the end of the acceptor stem (reviewed in Hinnebusch, A. G. (2000). Mechanism and Regulation of Initiator Methionyl-tRNA Binding to Ribosomes. In Translational Control of Gene Expression, N. Sonenberg, Hershey, J. W., and Matthews, M., ed. (Cold Spring Harbor: Cold Spring Harbor Press), pp. 185-243). eIF2, GTP and Met-tRNAi form a ternary complex that is required for binding to the ribosome. Subsequently, eIF2 leaves the ribosome as an eIF2. GDP binary complex, leaving the initiator tRNA on the ribosome. In order to recycle to its active form and bind another Met-tRNAi, eIF2.GDP must be converted to eIF2.GTP through a guanylate exchange reaction catalyzed by eIF2B. eIF2 has a 400-fold greater affinity for GDP than for GTP. The exchange of tightly bound GDP for GTP requires eIF2B, which is in limiting concentrations and present at 15-25% of the amount of eIF2 (reviewed in Hershey, J. W. B. (1991) Annu.Rev. Biochem. 60, 717-755; Hinnebusch, 2000; Jackson, 1991; Trachsel, H. (1996). Binding of initiator methionyl-tRNA to ribosomes. In Translational Control, J. W. B. Hershey, M. B. Mathews and N. Sonenberg, eds. (Cold Spring Harbor: Cold Spring Harbor Laboratory Press), pp. 113-138).

The recycling of eIF2 is inhibited by phosphorylation of its Alpha -subunit. Phosphorylation of the Alpha -subunit of eIF2 at the serine 51 residue is carried out by a family of eIF2 Alpha kinases. Phosphorylated eIF2(Alpha P).GDP binds much more tightly to eIF2B than eIF2.GDP and renders eIF2B non-functional. Thus, once the amount of phosphorylated eIF2 exceeds the amount of eIF2B, the protein synthesis is shut-off ( reviewed in Hinnebusch, 2000). Four eIF2 Alpha kinases have been identified in mammals: the dsRNA-dependent eIF2 Alpha kinase (PKR), the GCN2 protein kinase, the endoplasmic reticulum (ER) resident kinase (PERK) and the heme-regulated eIF2 Alpha kinase (HRI). Although they all inhibit protein synthesis by phoshorylation of eIF2 Alpha, it is predicted that differential effects may be produced as a consequence of their tissue distributions and the signals to which they respond. These eIF2 Alpha kinases share extensive homology in their kinase catalytic domain (Berlanga, et al. (1998) J. Biol. Chem. 273, 32340-32346; Chen, et al. (1991b). Proc. Natl. Acad. Sci. USA 88,7729-7733; Chong, et al.(1992)EMBO J. 11, 1553-1562; Harding, et al. (1999) Nature 397, 271-274; Meurs, et al. (1990) Cell 62, 379-390; Ramirez, et al. (1991) Mol. Cell. Biol. 11, 3027-3036; Shi, et al. (1998) Mol. Cell. Biol. 18, 7499-7509). However, each of the eIF2 Alpha kinases has a unique regulatory domain, which acts as a sensor for specific stimuli. PKR is ubiquitously expressed, induced by interferon, and regulated by dsRNA through two N-terminal dsRNA-binding domains (reviewed in Kaufman, R. J. (2000). Double-stranded RNA-activated protein kinase, PKR. In Translational Control of Gene Expression, N. Sonenberg, Hershey, J. W.; Matthews, M., ed. (Cold Spring Harbor: Cold Spring Harbor Press), pp. 503-528). GCN2 is highly expressed in the liver and brain (Berlanga, et al. (1999) Eur. J. Biochem. 265, 754-762; Sood, et al.(2000) Genetics 154, 787-801), and is activated under conditions of amino acid starvation through the C-terminal domain, which contains a His-tRNA synthase-like sequence (reviewed in Hinnebusch, 1996). PERK is highly expressed in secretory tissues, particularly the pancreas, and is activated by ER stress. PERK contains a lumenal domain which is similar to the sensor domain of the ER-stress kinase Irel (reviewed in Ron, et al. (2000). PERK and translational control by stress in the endoplasmic reticulum. In Translational Control of Gene Expression, H. J. W. B. a. M. M. B. Sonenberg N., ed. (Cold Spring Harbor: Cold Spring Harbor Laboratory Press), pp. 547-560; Harding et al., 1999; Shi et al., 1998). HRI is expressed predominantly in erythroid cells and is regulated by heme, the prosthetic group of hemoglobin, through the two heme-binding domains located in the N-terminus and the kinase insertion (reviewed in Chen, J.-J. (2000). Heme-regulated eIF-2a kinase. In Translational Control of Gene Expression, J. W. B. Hershey, M. B. Mathews and N. Sonenberg, eds. (Cold Springs Harbor: Cold Spring Harbor Laboratory Press), pp. 529-546; Chefalo, et al. (1998) Eur. J. Biochem. 258, 820-830; Rafie-Kolpin, (2000) J. Biol. Chem. 275,5171-5178).

HRI has been extensively studied biochemically. It is well documented that protein synthesis in intact reticulocytes and their lysates is dependent upon the availability of heme. In heme-deficiency, inhibition of protein synthesis correlates with the activation of HRI (reviewed in Chen, 2000 and Clemens, M. J. (1996). Protein kinases that phosphorylate eIF-2 and eIF-2B, and their role in eukaryotic cell translational control. In Translational Control of Gene Expression, J. W. B. Hershey, M. B. Mathews and N. Sonenberg, eds. (Cold Springs Harbor: (Cold Spring Harbor Laboratory Press)), pp. 139-172). Expression of HRI in insect Sf9 cells causes global inhibition of protein synthesis (Chefalo, et al. (1994) J. Biol. Chem. 269, 25788-25794). In addition, baculovirus-expressed HRI is a hemoprotein whose activity is regulated by micromolar concentrations of hemin both in vitro and in vivo (Chefalo et al., 1998; Chefalo, 1994).

HRI contains two distinct heme-binding sites. Heme bound to the N-terminal domain is stable and copurifies with HRI to homogeneity. In contrast, heme binds to the kinase insertion domain reversibly and inhibits HRI kinase activity upon binding, thereby regulating HRI activity according to intracellular heme concentrations (Chefalo et al., 1998; Rafie-Kolpin et al., 2000). HRI protein, activity and mRNA are detected predominantly in red blood cell precursors (Crosby, et al. (1994) Mol. Cell. Biol. 14,3906-3914), and HRI mRNA levels increase during erythroid differentiation of mouse rythroleukemic (MEL) cells (Crosby et al., 1994). Small amounts of HRI mRNA are also found in non-erythroid tissues, but no evidence of HRI protein expression has been reported (Berlanga et al., 1998; Mellor, et al. (1994) J. Biol. Chem. 269, 10201-10204). The mechanism by which this is achieved, and means for controlling inhibition, or stimulating inhibition of HRI protein expression, are still not well characterized.

HRI was first identified as an inhibitor of protein synthesis in reticulocytes in 1960 (reviewed in Chen, J.-J. (1993). Translational regulation in reticulocytes: The role of heme-regulated eIF-2 Alpha kinase. In Translational Control of Gene Expression 2, J. Ilan, ed. (New York: Plenum Press), pp. 349-372). Although HRI has been extensively studied biochemically, its physiological function in a whole animal was unknown. To date no disease in human or mouse is known to be associated with mutations in the HRI gene. Yet, orthologs of HRI appear widely conserved from fish to humans, suggesting an important role for HRI.

It is therefore an object of the present invention to provide an animal model in which HRI is knocked out.

It is a further object of the present invention to provide methods for expression of HRI in mammalian cells and screening of compounds for alteration of expression.

It is still another object of the present invention to provide methods of use of the identified compounds to inhibit cell proliferation, by inhibiting protein synthesis.

### SUMMARY OF THE INVENTION

The targeted disruption of the gene encoding the heme-regulated eIF2_kinase (HRI) in mice establishes that HRI, which is expressed predominantly in erythroid cells, regulates the synthesis of both alpha and beta globins in red blood cell precursors by inhibiting the general translation initiation factor eIF2. This inhibition occurs when the intracellular concentration of heme declines, thereby preventing the precipitation of excess globin polypeptides. In iron deficient HRI−/− mice, globins devoid of heme aggregated within red blood cells and their precursors, resulting in a hyperchromic, hemolytic anemia with compensatory erythroid hyperplasia in the marrow and spleen. Thus, HRI is a physiological regulator of gene expression in the erythroid lineage and acts both as a sensor of heme and an effector controlling the initiation of translation. Accordingly, regulation of HRI can be used to control proliferation of cells, especially cells of hematopoietic origin, using compositions identified by efficacy in testing of the HRI knockout mice.

### BRIEF DESCRIPITION OF THE DRAWINGS

FIG. 1 is a schematic of HRI cDNA indicating the locations of the eleven domains, the HRI specific insertion region, and the three peptides previously sequenced and identified as unique to HRI: P-52, corresponding to amino acids 454 to 467, containing Asp-Phe-Gly, which is the most highly conserved short stretch in catalytic domain VII of protein kinases; P-74, corresponding to amino acids 506 to 525, containing the conserved amino acid residues Asp-(Met)-Tyr-Ser-(Val)-Gly-Val (SEQ ID NO: 1) found in catalytic domain IX of protein kinases, and P-56, corresponding to amino acids 166-178, as shown in U.S. Pat. No. 5,525,513 to Chen, et al.

FIG. 2 is a schematic of the targeted disruption of the HRI gene: top is the HRI wild-type locus; middle is the targeting construct; and bottom is the targeted homologous recombination of the HRI locus before and after Cre-mediated excision of the neomycin resistance gene.

FIGS. 3A and 3B are graphs of the loss of heme-dependent globin synthesis in HRI−/− reticulocytes over time (minutes) in the presence of heme (H) or cycloheximide (CHX), and control (C) untreated reticulocytes. Samples were taken every 30 minutes to be analyzed for globin synthesis.

FIGS. 4A-D are graphs showing the hematological analysis of HRI +/+ mice in iron deficiency. The RBC number and the red blood cell indices, MCV, MCH, Hb, and HCT were obtained from blood collected from the tail veins. Time courses of these changes from day 17 to 84 are shown. Four to six mice were used for each group. The differences in MCV and MCH and RBC number are statistically significant on low iron diet (p (0.001 for all these three parameters).

FIGS. 5A and 5B are graphs of the decreased survival of HR −/− mice in phenylhydrazine-induced hemolysis (FIG. 5A) and the percentage survival at different dosages at day 6 after initial phenylhydrazine injection (FIG. 5B).

FIGS. 6A and 6B are schematics of models of the role of HRI duringerythroid differentiation. FIG. 6A shows regulation of alpha and beta globin synthesis by HRI and heme. FIG. 6B shows altered hematological response of HRI−/− mice to iron deficiency.

### DETAILED DESCRIPTION OF THE INVENTION

As described in U.S. Pat. No. 5,525,513 to Chen, et al., HRI cDNA was cloned from a lambda Zap II cDNA library of rabbit reticulocytes. As described in more detail below, this cDNA is highly homologous to human DNA encoding HRI and has been used to obtain a clone encoding the human HRI, as well as HRI from other species such as mouse (although there appears to be slightly greater homology between rabbit and human than between rabbit and mouse HRI). The rabbit HRI cDNA contains 2729 nucleotides and encodes 626 amino acids. The nucleic acid sequence has been deposited in the Gene Bank data base (accession No. M69035). In vitro translation of HRI mRNA transcribed from HRI cDNA yields a 90 kDa polypeptide with eIF-2Alpha kinase activity. This 90 kDa polypeptide is recognized by anti-HRI monoclonal antibody. These properties are characteristic of authentic HRI.

The open reading frame sequence of the HRI cDNA contains all eleven catalytic domains of protein kinases with consensus sequences of serine/threonine protein kinases in conserved catalytic domains VI and VIII. The HRI cDNA also contains an insert of approximately 140 amino acids between catalytic domains V and VI. The HRI cDNA coding sequence has extensive homology to GCN2 protein kinase of S. cerevisiae and to human double stranded RNA-dependent eIF-2Alpha kinase. It therefore is believed that GCN2 protein kinase may be an eIF-2Alpha kinase in yeast. Recently, it has been shown that phosphorylation of e2F-2Alpha by GCN2 is required for the translational control of yeast GCN4, Dever, et al., Cell 28, 585-596 (1992). In addition, HRI has an unusually high degree of homology to three protein kinases, Nim A, Wee1 and CDC2, which are involved in the regulation of the cell cycle.

Expression and Characterization of HRI from the Isolated cDNA

The 5′ untranslated leader sequence of the HRI cDNA was replaced by the use of PCR to introduce a unique Ncol site (CCATGG) at the initiating methionine (nt 113), followed by ligation of the coding sequence to a vector containing the tobacco mosaic virus (TMV) untranslated leader sequence which was engineered to provide both the initiating methionine and 3′-terminal Ncol site. The introduction of the Ncol site changes the second amino acid of HRI from leucine to valine, constituting a conservative substitution.

Linearized HRI cDNAs were transcribed using T7 polymerase. In vitro translation of HRI mRNA (40 ìg/ml) was carried out in the presence of [³⁵S]-methionine as described by Promega using nuclease-treated reticulocyte lysates or wheat-germ extracts. Protein kinase assays were carried out in 40 ìl reactions with 10 mCi of [g-³²P]ATP (3,000 Ci/mmol), 1.5 ìl of translational mixture and purified rabbit eIF-2 (1 ìg) as indicated, at 30_C (reticulocyte lysate) or 25_C (wheat germ extract) for 10 min as described by Chen, J. -J., et al., J. Biol. Chem., 264:9559-9564 (1989).

In vitro transcription and translation were carried out in order to determine the apparent molecular size of the protein encoded by the HRI cDNA and to test for protein kinase activity. Translation of all five HRI clone mRNAs in a nuclease-treated rabbit reticulocyte lysate yielded a predominant 90 kDa product as observed by SDS-PAGE.

The nucleotide sequence data demonstrate that the 5′ untranslated leader sequence is extremely G-C rich with the potential to form significant secondary structure. Secondary structure at the 5′-terminus of mRNAs is known to diminish mRNA translational efficiency. The HRI mRNA was not translatable in a wheat germ extract. Unlike the reticulocyte lysate, the wheat germ extract does not contain an endogenous HRI enzyme; therefore, expression of the HRI protein in the wheat germ system should facilitate analysis of kinase activity in the HRI translation products. The translational efficiency of mRNA transcripts can be increased by the use of untranslated leader sequences of some plant viral RNAs such as TMV have been shown to act in cis by Gallie, et al., (1987) Nucl. Acids Res., 15, 8693-8711, and Gehrke, L. and Jobling, S. A., (1990) In: McCarthy, JEG Post-Transcriptional Regulation of Gene Expression, Series H: Cell Biology, ed. Tuite, M. (Springer Verlag, Berlin), Vol. 49, pp. 389-398. Accordingly, the G-C rich HRI untranslated leader sequence was replaced with that of TMV. The chimeric TMV-HRI mRNA was translated with approximately tenfold greater efficiency than HRI mRNA in the reticulocyte lysate, and translation in the wheat germ extract was clearly evident. In all cases, the translated product of HRI mRNA migrated slightly faster than authentic purified phosphorylated HRI on SDS gel electrophoresis. This slight difference in mobility is most likely due to a lower level of phosphorylation in the translation products.

To determine whether the translational product derived from the mRNA of HRI cDNA is an eIF-2Alpha kinase, a small portion of the total translation mixture was incubated with purified rabbit reticulocyte eIF-2 and [g-³²P] ATP in the absence of added hemin under protein kinase assay conditions and analyzed by SDS-gel electrophoresis.

The results show that translational products of HRI 2A and HRI 2B mRNAs have enhanced eIF-2Alpha kinase activity as compared to the control in the absence of added mRNA. It should be emphasized that under the kinase assay conditions (final hemin concentration of 0.75 ìM) the activity of newly synthesized HRI exceeds the low activity of endogenous pre-formed HRI in the nuclease-treated lysate and makes it possible to detect enhanced phosphorylation of eIF-2Alpha. In the absence of added purified rabbit eIF-2, only slight phosphorylation in the region of eIF-2Alpha is observed. Furthermore, the HRI polypeptide synthesized in the wheat-germ extracts exhibits eIF-2Alpha kinase activity as does purified HRI. It should be noted that there is no mammalian eIF-2Alpha kinase activity in the wheat-germ extracts, and the purified reticulocyte HRI phosphorylates purified wheat germ eIF-2Alpha very inefficiently. In addition, the 90 kDa polypeptide expressed from HRI cDNA is immunoprecipitated by monoclonal antibodies to HRI.

Isolation of cDNA Encoding HRI in Other Mammalian Species

DNA nucleotide sequence data were analyzed in part using CAD Gene™ software for the MacIntosh™ computer, provided by the Genetic Technology Corporation, Cambridge, Mass. The amino acid sequences of dsRNA-dependent eIF-2Alpha kinase (dsI) of rabbit and human are 83% similar and 76% in identity. Similar or higher degree of homology of initiation factors (eIF-2Alpha, and eIF-2a eIF-4A, eIF-4E, EF-1a) between human and rabbit has been demonstrated. The predicted homology of HRI between human and rabbit is greater than 80%. Accordingly, the sequence encoding HRI in human or other species can be isolated by hybridization under standard conditions such as those outlined by Maniatis, et al., (1989) Molecular Cloning. A Laboratory Manual, from a library prepared from reticulocytes of the other species. The isolated sequence can then be expressed in the same manner as the HRI cDNA isolated from rabbit reticulocytes.

HRI cDNA contains an insertion of approximately 140 amino acids between catalytic domains V and VI (amino acids 276 to 413). Similar large inserts have been reported for subclass III and IV receptor tyrosine kinases, which include the PDGF receptor, the CSF-1 receptor and the c-kit proto oncogene product, in which the kinase domains are divided into two halves by insertion of up to 100 mostly hydrophilic amino acid residues, as reviewed in Ullrich, A. and Schlessing, J., (1990) Cell, 61:203-212. Since kinase insertion sequences are highly conserved among species for each specific receptor, the kinase insert may play an important role in the action of receptor kinases. Indeed, the PDGF receptor kinase insert contains an autophosphorylation site (Tyr-751), and mutation of Tyr-751 to Phe or Gly blocks association of the PDGF receptor with phosphatidylinositol kinase and three other cellular proteins. In the case of HRI, heme binds to HRI and regulates its kinase activities. It is believed that the kinase insertion sequence of HRI is involved in the binding of heme and the regulation of the autokinase and eIF-2Alpha kinase activities.

Inhibition of Cell Proliferation and Differentiation and Viral Activity and the Induction of Differentiation using HRI or dsI

Since HRI is a potent inhibitor of protein synthesis, it is anti-proliferative in nature and should be useful in the treatment of various cancers in which uncontrolled cell growth persists, for example chronic myelogenous leukemia. HRI should also be useful in treatment of other proliferative disorders such as psoriasis.

Initiation of protein synthesis can also be regulated by another eIF-2Alpha kinase which is activated by double-stranded RNA (dsI). Both HRI and dsI phosphorylate eIF-2Alpha at the same site. However, HRI and dsI are different molecules. dsI is induced by interferon and represents an interferon mediated response to viral infection. However, mechanisms of inactivating dsI have evolved in various viruses to undermine the anti-viral action of dsI. Since HRI and dsI are both eIF-2 Alpha kinases, both should be anti-viral in nature. However, mechanisms of inactivating viruses by dsI should not similarly affect HRI activity. Therefore, when introduced into the proper target cell, HRI may be as potent or more potent than dsI as an anti-viral agent.

Since HRI is expressed normally only in very small quantities, in the cytoplasm, and during specific periods of erythroid differentiation, small quantities of the protein are expected to be effective in inhibiting protein synthesis, inducing differentiation, and inhibiting infection by viruses and parasites.

The HRI, expressed from the cDNA, preferably of the same species as the cells to be treated, can be administered topically, by injection, or via implant to the cells or patient to be treated. Appropriate pharmaceutical compositions and methods for administration and use thereof are well known to those skilled in the art. The HRI can be expressed in any suitable mammalian expression system, using known technology, under the control of appropriate enhancers and promoters.

Alternatively, the cells to be treated are “infected” with the sequence encoding the HRI. In the preferred embodiment, this is accomplished by inserting the HRI sequence into a retroviral vector with which the cell is then infected. For example, a retroviral vector for gene transfer and expression of HRI cDNA can be constructed using as the backbone of the retroviral vector the LNCX vector described by Miller and Rosman (Miller, A. D., and Rosman, G. J. (1989) BioTechniques 7:980-990). It contains human cytomegalovirus (CMV) immediate early gene promoter and enhancer. HRI cDNA containing TMV-leader sequence is introduced into the LNCX vector through a polylinker region downstream from the CMV promoter.

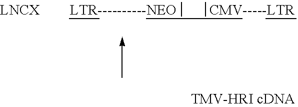

Gene transfer by retroviral vector can also be achieved by transfection by a viral vector, using the method of Wilson, J. M, Biriuyi, L. U., Salomon, R. U., et al. Transplantation of Vascular Grafts Lined With Genetically Modified Endothelial Cells. Science, 244:1344-1346 (1980), or a plasmid transfer technique, as described by Felgner, P. L., Galik, T. R., Holmer, et al. Lipofection: An Efficient, Lipid Mediated DNA-Transfection Procedures. Proc. Natl. Acad. Sci., 84:7413-7417 (1987), the teachings of which are incorporated herein by reference.

Specifically, cells are harvested, grown to subconfluence (60-70%) and incubated with a replication defective murine Moloney leukemia retroviral vector. The DNA sequence for HRI is inserted into the viral genome and is under the promoter control of the viral long-terminal repeats (LTR's). The infected cells are trypsinized, resuspended in saline containing penicillin (100 U/ml) and streptomycin (100 ìg/ml) and transplanted into the patient requiring treatment. The presence of HRI in the culture medium or the site of transplantation can be determined by radioimmunoassay.

Construction of Deletion Mutants of HRI cDNA that are Insensitive to Heme, Less Species Specific or Overexpressed

Deletion mutants of HRI cDNA which are not sensitive to regulation by heme can be constructed, based on the prediction that the heme-binding region is found within the HRI-specific insert discussed above and/or in the 170 N-terminal amino acids. This heme-insensitive HRI may be more effective than native HRI in its anti-viral and anti-proliferative action.

Deletion mutants of HRI cDNA can also be constructed which are less species specific. There is greater than 80% homology between species (86% between human and rabbit dsI). The primary area of species variation is in domain V. Methods for constructing and screening for these mutations are known to those skilled in the art.

Construction of HRI Double Knockout Mice

In order to elucidate the physiological role of HRI in mammals in the context of a whole animal, the HRI gene was disrupted in mouse ES cells. HRI−/− mice appear to be normal, are fertile and present no gross abnormality of hematological parameters. However, upon depletion of the intracellular pool of heme by diet-induced iron deficiency, the adaptive response of wild-type mice, characterized by red blood cell hypochromia and microcytosis, was dramatically altered. In HRI−/− mice, red blood cell size remained normal with hyperchromic rather than hypochromic anemia. Globins devoid of heme aggregated as inclusions within the red blood cell and its precursors resulted in a hemolytic anemia with compensatory erythroid hyperplasia in marrow and spleen. In reticulocytes of HRI−/− mice, eIF2a phosphorylation was dramatically reduced. The global rate of protein synthesis increased by seven fold and became independent of heme. Together, these results establish the physiological role of HRI in balancing the synthesis of a and B globins with the availability of heme in red blood cell precursors and provide the evidence, in the context of a whole animal, that translational control of gene expression plays a key role in the processes of somatic cell differentiation. These animals (heterozygotes or homozygotes) can therefore be used to screen for compounds which alter HRI expression or activity, or mimic HRI activity, and can therefore be used to alter cellular proliferation and kinase activity. Gene expression, cellular phenotype, eIF-2 phosphorylation, or kinase activity toward eIF-2, are examples of easily assayed conditions that can be used in/as methods to screen for compounds that affect HRI related processes.

Experimental Procedures

Targeted Disruption of the Murine HRI Gene

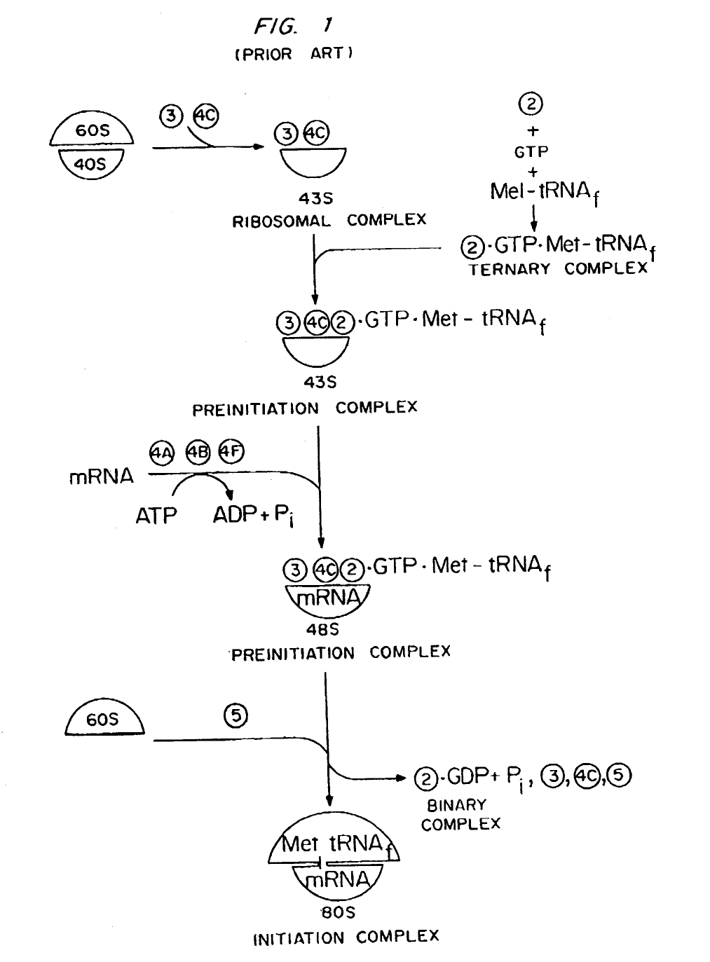

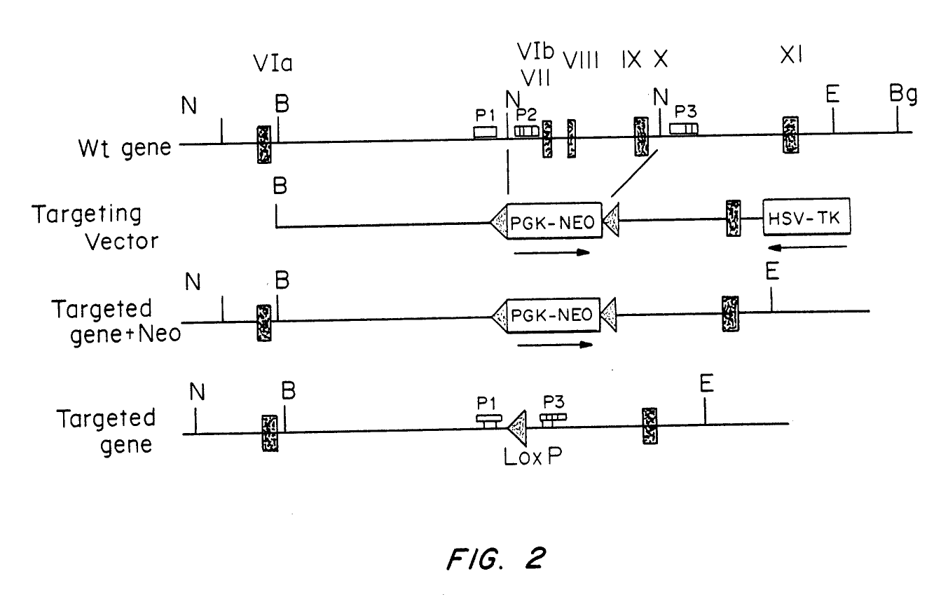

A lambda FixII phage genomic DNA library of a mouse strain 129/SV (Stratagen) was screened for the HRI gene using the entire coding sequence of rabbit HRI cDNA (Chen et al., 1991) as a probe. After screening 750,000 recombinant phages, three positive clones were isolated. Clone 19, which contains five exons, was used for targeting construct preparation. The 5 KB fragment containing three exons of HRI (encoding kinase domains VIb to X) was excised with Nde I, and replaced with floxed PGK-Neo (FIG. 1A). HSV-TK was ligated to the EcoR V site. The HRI targeting construct was linearized with Sal I and electroporated intoembryonic stem (ES) cells. Of two homologous-recombined ES clones, one clone with a normal karyotype was injected into blastocysts to produce chimeric mice and subsequently the heterozygous HRI+/− mice. Heterozygotes were crossed with the GATA-1/Cre mice to remove the PGK-Neo gene in the germline (Mao, et al. (1999) Proc Natl Acad Sci U S A 96, 5037-42). These PGK-Neo minus HRI+/− heterozygotes were interbred to generate HRI−/− homozygotes. Genotyping was performed by PCR of the tail DNA. Two PCR reactions were performed to determine the genotypes. PCR reaction 1 was carried out with primers 1 (5′AGCTCCACCCTGACGATCTA3′) (SEQ ID NO:2) and 2 (5′ATGTGCAGGGCTGAAGAGAT3′) (SEQ ID NO:3), and PCR reaction 2 with primers 1 and 3 (5′CATGCTGGGGGTCAAATAGT3′) (SEQ ID NO:4), as illustrated in FIG. 2. The conditions for PCR were denaturation at 95_C for 2 minutes, followed by 30 cycles of amplification (denaturing at 95_C for 1 minute, annealing at 60_C for 1 minute and extension at 72_C for 2 minutes), and a subsequent extension at 72_C for 10 minutes, using Taq polymerase (Perkin Elmer, USA), 5 pmol of each primer, 2.5 mM MgCl2 and 400 _M dNTPs.

Such chimeric mice, heterozygotes (HRI -/+), and/or homozygotes (HRI −/−) are useful animal model systems used in screening compounds affecting HRI activity.

Cloning of the Mouse HRI cDNA

For the characterization of the phenotype of HRI knockout mice, the mouse HRI cDNA was cloned from mouse erythroleukemic cells (MEL). A lambda gtll cDNA library of DMSO-induced MEL cells (Andrews, et al. (1993a) Nature 326, 722-728) was screened with the entire coding sequence of rabbit HRI cDNA (Chen et al., 199 lb). Several positive clones were isolated, and the longest partial mouse HRI cDNA was amplified by PCR from the recombinant lambda phage DNA (clone 13). The nucleotide sequence of clone 13 contains the sequence encoding amino acids 391 to 619 plus 675 nucleotides of 3′T UTR. To obtain the full-length mouse HRI cDNA, the mouse EST database was searched for the HRI sequence 5′ upstream of clone 13. An EST sequence which contained the first initiating methionine from mouse muscle cell line C2C12 (AA692384) was found. The mouse N-terminal HRI cDNA sequence was then amplified from MEL cDNA by PCR using the 5′ primer with sequence right before the first ATG (5′GGAATTCCTATCCACGCTCCGAAC GGC A3′) (SEQ ID NO:5) and the 3′ primer from the 5'sequence of the clone 13 (5° CCCAAGCTTGCAACAC TGGCCATAACATAG 3′) (SEQ ID NO:6) with 10% DMSO in the PCR reaction to overcome the secondary structure in the 5′ of HRI cDNA. The MEL cDNA template used for PCR was reversed-transcribed from the mRNA of DMSO-induced MEL cells with HRI specific primers (HRI 3′ Primer). The assembled full-length mouse HRI cDNA encodes 619 amino acids and exhibits high homology (82%) to rabbit HRI.

Production of Anti-HRI Polyclonal Antibodies

Antibodies against the N-terminus and the kinase insertion sequence of the mouse HRI were produced to help characterize the HRI knockout mice. The mouse N-terminal 139 amino acids and the kinase insertion sequence (amino acids 241-405) were subcloned into pET 28a vector and expressed in E. coli BL-21 cells as a fusion protein with a N-terminal (His)6-tag and the TEV-protease cleavage site to facilitate the purification. His-tagged N-terminus and KI were purified over Ni++-agarose column. The His-tags of the purified proteins were removed by TEV-protease and the HRI domains were then used to immunize the rabbits. The antibodies were prepared by HTI Bio-Products (Ramona, Calif.).

Production of Mouse Reticulocytes, Protein Synthesis, Protein Kinase Assays and Western Blot Analysis

Mice were injected with phenylhydrazine at 40 mg/kg on days 0, 1 and 3 to induce hemolysis, and the subsequent erythropoiesis and release of reticulocytes into the blood stream. Blood samples were collected by heart puncture on day 7 when the reticulocyte counts were 85-95%. Reticulocytes were washed twice with ice-cold phosphate-buffered saline supplemented with 5 mM glucose. For protein synthesis assays, reticulocytes were resuspended in DMEM (2×10⁸ cells/ml) with 1/10 concentration of methionine plus 2% dialyzed fetal bovine serum and preincubated for 30 min at 37_C for recovery. The reticulocytes were then treated with hemin (40 ìM) or cycloheximide (2 ìM) as indicated, and labeled with [³⁵S]-methionine (5_Ci, 3000 Ci/mmol). 200_l of cell suspension were lysed in SDS-sample buffer containing EDTA (1 mM) and NaF (50 mM) to prevent further phosphorylation or dephosphorylation of HRI and eIF2aP. Incorporation of the [³⁵S]-methionine in the globin chains and other proteins was analyzed by 15% SDS-PAGE, and subsequent transfer to nitrocellulose membranes. Protein synthesis in reticulocytes was quantitated by scintillation counting of the nitrocellulose strips containing globin chains. Phosphorylation of HRI and eIF2a in isolated reticulocytes were analyzed by western blot analysis following 7.5% and 12% SDS-PAGE respectively. In vitro protein kinase assays using the endogeneous eIF2 as a substrate were performed as described by Chen, et al. (1989) J. Biol. Chem. 264, 9559-9564.

Diet-Induced Iron Deficiency and Hematological and Pathological Analyses

A state of iron deficiency was induced by placing newborn mice after weaning on low iron diet containing 5ppm Fe. Some of the littermate were fed normal diet containing 196 ppm Fe as controls. Four to six mice of each sex were used in each of four groups, Wt mice on a normal diet, Wt on an iron-deficient diet, knockout mice (Ko) on a normal diet, Ko on an iron-deficient diet. Hematological analysis of the peripheral blood collected from the tail vein was performed biweekly by the Division of Comparative Medicine at MIT using a Hemavet(800 instrument (CDC Technologies Inc.).

Cell morphology of the peripheral blood was examined by Wright-Giemsa staining of the blood smears. Reticulocyte counts were manually counted on smears stained with new methylene blue as described (Beutler, E. (1983). Heinz body staining; Blood, marrow and iron staining. In Hematology, W. J. Williams, Beutler,E., Erslev, A. J., and Lichman, M. A., ed.: McGraw-Hill Book company), pp. 1603-1604). Heinz bodies in the blood samples were determined by staining live cells with crystal violet (Beutler, 1983). Tissues were fixed in formalin and processed for paraffin embedding and sectioning using standard procedures by the MIT Division of Comparative Medicine. Electron microscopy was performed on Karnofsky's fixed, EDTA-anticoagulated blood using standard methodologies in the Department of Pathology, Children's Hospital.

Results

Cloning of the Murine HRI Gene and Generation of HRI−/− Mice

A 19 Kb mouse genomic DNA fragment was cloned from a phage library using a rabbit HRI cDNA as a probe (FIG. 2: Targeted Disruption of the HRI Gene.(A) HRI wild type locus (top), targeting construct (middle), and targeted homologous recombination at the HRI locus before and after Cre-mediated excision of the Neomycin resistance gene (bottom)). The 5 KB DNA fragment containing three exons (marked by the filled rectangles) in HRI locus was replaced with PGK-neo in the targeting construct that also has HSV-TK as a negative selection marker. Genotyping of the targeted disrupted mice was performed by PCR. The primers are indicated as P1, P2 and P3. P1 and P2 were used for amplification of the HRI+/+ DNA of 625 bp. P1 and P3 were used for the amplification of HRI−/− DNA of approximately 1000 bp. PCR reactions were done separately for both sets of the primers and the PCR products of both reactions for the DNA of the same mouse were run together.

Total RNA was isolated from E20 fetal livers of HRI +/+, and −/− embryos. The expression of HRI mRNA was determined by RT-PCR. Based on the known organization of the HRI protein, this genomic fragment contains 5 exons of the mouse HRI gene. These exons encode the second conserved kinase lobe of HRI with kinase domains VIa to XI and the entire C-terminus. The HRI gene was mapped to the very distal end of mouse chromosome 5, which corresponds to human chromosome 7p13q. A database search of the human genome confirmed the location of the HRI gene. So far, no known human or mouse disease is linked to these chromosomal loci. The targeting construct was prepared by replacing a 5 Kb DNA segment containing the 3 exons that encode kinase catalytic domains VIb to X with the Neomycin phosphotransferase gene under the control of the phosphoglycerate kinase promoter (PGK-Neo). These three exons were deleted since they encode a region essential for the kinase activity of HRI. Thus, in the event that an HRI protein bearing this internal deletion would be produced from the targeted HRI gene, it would be inactive. Correct targeting of the mouse HRI gene was confirmed by PCR analysis of tail DNA.

HRI−/− mice are viable and fertile without gross morphological abnormalities. Before characterizing the phenotype further, HRI mRNA was examined in +/+, and −/− mice by RT-PCR of RNA isolated from day 19.5 embryonic livers. RNA from +/+ fetal livers produced a diagnostic 1867 bp DNA fragment containing the entire coding sequence. RNA from −/− fetal livers produced a smaller DNA fragment of the expected size of 1463 bp with correct splicing of the mRNA over the 3 deleted exons of the targeted HRI gene. Since HRI protein is expressed predominantly in the erythroid lineage and most abundantly in reticulocytes, the expression of HRI protein in reticulocytes of +/+, +/− and −/− mice was examined by Western blot analysis using antibodies directed against the N-terminal 138 amino acids of mouse HRI, whose encoding exons were not removed by genomic targeting. The levels of HRI protein expression in the lysates of 1×10⁶ reticulocytes of HRI +/+, +/− and −/− mice were examined by western-blot analysis . The IF2Alpha kinase activity of HRI in reticulocyte lysates was determined using in vitro protein kinase assays. The extent of the eIF2a phosphorylation was determined by western-blot analysis using antibody specific to the phosphorylated eIF2a. Protein synthesis was carried out by labeling the equivalent HRI +/+ or −/− reticulocytes (2×10⁸ reticulocytes/ml) with [³⁵S] methionine for 90 minutes. Samples (3×10⁵ reticulocytes) were taken every 15 min to be analyzed for rate of protein synthesis and eIF2a phosphorylation as indicated. Globin synthesis in HRI+/+ Reticulocytes amd loss of heme-dependent globin synthesis in HRI−/− Reticulocytes were measured. Reticulocytes from HRI+/+ and HRI−/− mice were incubated with [³⁵S]-methionine for 90 minutes in the presence of hemin (H), or cycloheximide (CHX). “C” denotes untreated control reticulocytes. Samples were taken every 30 min to be analyzed for the rate of globin synthesis.

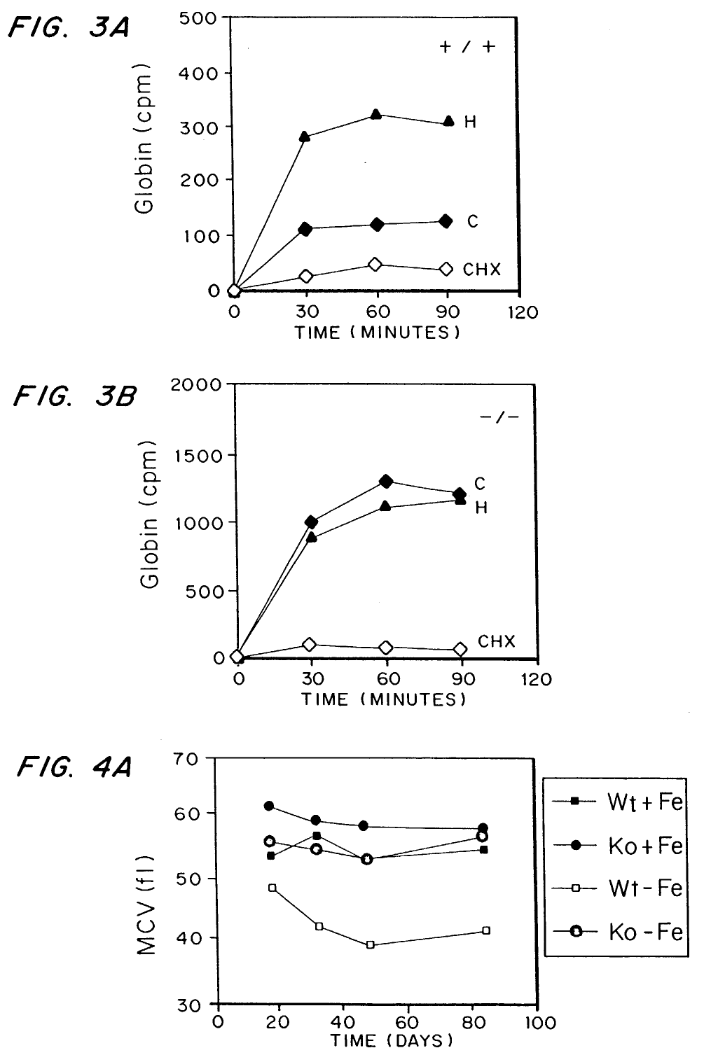

HRI protein was detected in +/+ and heterozygote mice but not in −/− homozygotes (FIG. 3). These data suggest that the truncated HRI protein encoded by the targeted gene is unstable, and demonstrate that HRI null mice are obtained.

Mild Macrocytosis and Hyperchromia in Otherwise Normal HRI−/− Mice in the Absence of Stress

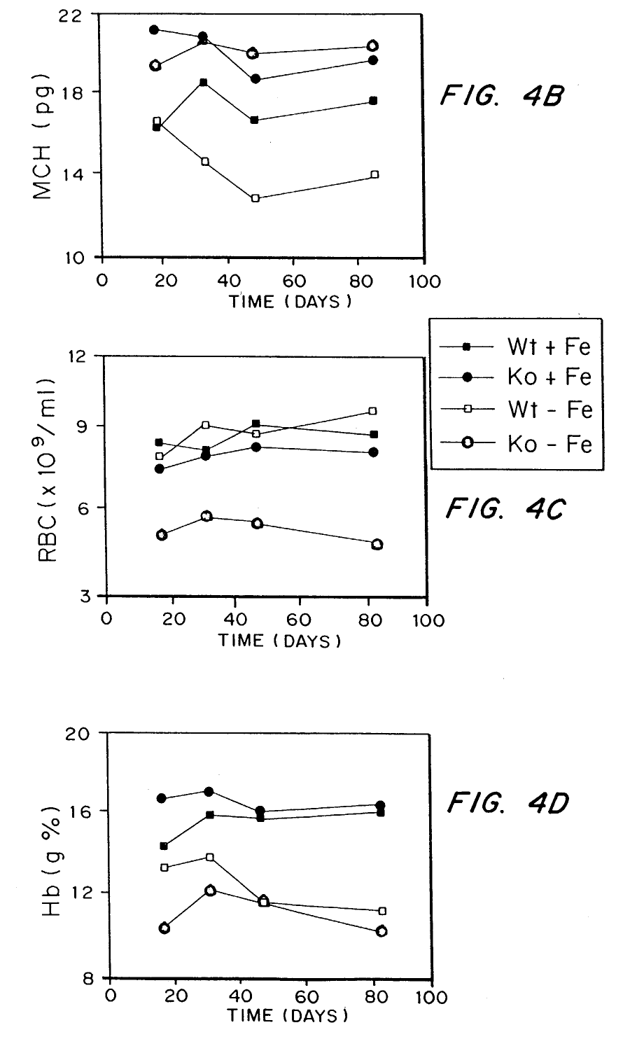

Only minimal abnormalities were detected in the hematological parameters of HRI−/− mice in the absence of stress. There was a slight increase in mean red blood cell volume (MCV) accompanied by a moderate but significant increase in the mean red blood cell hemoglobin (MCH) (FIG. 4). As hemoglobin concentration per red blood cell is normally tightly regulated, no case of elevated MCH, referred to as hyperchromia, has ever been reported in humans or other mammals. There was also an increase in the number of Heinz-body containing red blood cells in HRI−/− mice (see below).

Profound Decrease in eIF2_Phosphorylation in vivo in HRI−/− Reticulocytes

Although HRI is the predominant eIF2_kinase in reticulocytes and nucleated erythroid precursors, whether other ubiquitous eIF2 Alpha kinases such as PKR might substitute for HRI in HRI−/− mice. The extent of eIF2Alpha phosphorylation was determined by Western blot using an antibody specific to phosphorylated eIF2(DeGracia, et al. (1997) J. Cereb. Blood Flow Metab. 17, 1291-1302). The level of eIF2 Alpha phosphorylation in intact reticulocytes was dramatically decreased in HRI−/− homozygotes as compared to HRI+/+ mice (FIG. 2B) while total eIF-2 Alpha protein level was not altered significantly. The low-level eIF2(phosphorylation in HRI−/− reticulocytes comes from other eIF2(kinases such as PKR which is known to be present in rabbit reticulocytes (Farrell,P., Balkow, K., Hunt, T., Jackson, R. J., Trachso, H. (1977) Cell 11, 187-200). To assess HRI activity in vitro, eIF2 Alpha phosphorylation assays were performed with reticulocyte lysates in the presence of an excess of exogenous ATP, using endogenous eIF2 as a substrate. In vitro eIF2 Alpha phosphorylation occurred in reticulocyte lysates from +/+ mice Alpha and, to a lesser degree, in those Alpha of +/− heterozygotes. In contrast, little eIF2 phosphorylation was detected in the reticulocyte lysates of HRI−/− mice. These data indicate that most of the eIF2 kinase activity normally found in reticulocytes under these experimental conditions is abrogated in HRI−/− mice, and other eIF2Alpha kinases are unable to compensate for this loss of HRI function in reticulocytes. Induction, repression, or attenuation of HRI activity can therefore be easily assayed.

Increased Protein Synthesis in HRI−/− Reticulocytes and Abolition of the Regulation by Heme

Since HRI is an inhibitor of protein synthesis, the rates of protein synthesis in isolated, intact reticulocytes of HRI +/+ and −/− mice were examined by measuring the incorporation of [³⁵S]-methionine into newly translated proteins. Compared to +/+ mice, there was a marked increase in the rate of protein synthesis seen globally for all proteins.

This observation is consistent with the current knowledge that eIF2 is a general translational factor. However, this effect was most pronounced for both alpha and beta globins, which are the most abundantly expressed proteins in reticulocytes. Quantitation of [³⁵S]-methionine incorporation in the globin chains showed that there was a 7-fold increase in globin synthesis in HRI4- reticulocytes as compared to reticulocytes from HRI+/+ mice.

The effect of heme on protein synthesis in HRI +/+ and −/− reticulocytes was then examined. Incorporation of [³⁵S]-methionine into globins was increased to 208 % of control by pre-treatment of +/+ reticulocytes with 40_M hemin for 30 min prior to the addition of [^{S3}5]-methionine. Protein synthesis of both Wt and HRI−/− reticulocytes was inhibited equally well by cycloheximide, which inhibits translational elongation and is independent of HRI. In contrast, protein synthesis in HRI−/− reticulocytes was not affected by the addition of hemin.

Together, these results indicate that, in the absence of HRI, the steady state level of protein synthesis in reticulocytes is considerably increased in a heme-independent manner, with the main impact on the accumulation of excess alpha and beta globin chains, the most abundant proteins translated in these cells.

Alteration of the Hematological Response of HRI−/− Mice to Diet-Induced Iron Deficiency: Hemolytic Anemia with Erythroid Hyperplasia

The profound biochemical changes in HRI−/− reticulocytes described above contrast with the minor alterations in the hematology of the mice in the absence of stress. Since it was hypothesized that HRI may act as a heme sensor that regulates translation in red blood cell precursors, one would expect to be able to exacerbate the phenotype of HRI−/− mice under conditions in which HRI is activated, particularly in heme deficiency. The options to render mice heme-deficient for a long-term study are limited. Since the life span of mouse red blood cells is around 40-50 days (Hoffmann-Fezer, et al. (1993) Ann. Hematol. 67, 81-87), prolonged treatment of mice with the inhibitors of the heme biosynthetic pathway such as succinylaceton may cause side effects and is therefore not desirable. In order to induce heme-deficiency, mice were put on a low iron diet, since heme is synthesized by the insertion of iron into protoporphyrin IX via ferrochelatase during the last step of heme biosynthesis. In addition, iron-deficiency is a very common condition found in human and other mammals and it may, in fact, be that HRI ultimately exists to protect red blood cells from some of the major untoward consequences of iron-deficiency.

The normal adaptive response to iron deficiency is well characterized in human and mice. Both MCV and MCH decrease considerably accompanied by a mild decrease in red blood cell counts, resulting in the classical microcytic (low MCV) and hypochromic (low hemoglobin) anemia (FIGS. 4A-D, Wt−Fe). In HRI−/− mice, this physiological response to iron deficiency was dramatically altered (FIGS. 4A-D, Ko−Fe). MCV was only slightly decreased as compared to HRI−/− mice on a normal diet (FIGS. 4A-D, Ko+Fe), but was still similar to the Wt mice on a normal diet (Wt+Fe). Whereas MCH was slightly elevated compared to the Wt mice on a normal diet, red blood cell counts decreased considerably, resulting in a very unusual hyperchromic, normocytic anemia with decrease in red blood cell counts. As seen above, an increase in MCH (hyperchromia) has never been reported in humans nor mice. It is even more surprising to observe red blood cell hyperchromia in the setting of iron deficiency, when the normal response is characterized by a marked hypochromia. (Hematoxylin and Eosin stained sections of spleens from iron-deficient HRI+/+ and HRI−/− mice were analyzed from both Wt and HRI−/− mice both in normal and iron-deficient diets for 43 days. Peripheral blood smears were prepared from HRI +/+ and −/− mice maintained on an iron deficient diet for 33 days. No significant difference was observed between Wt and HRI−/− spleens, and blood in normal diet. Blood from mice of all four groups at day 92 after receiving low iron diet, was collected and stained with crystal violet for the presence of Heinz bodies.

Despite the hyperchromic RBCs, iron-deficient HRI−/− mice are anemic due to a profound decrease in RBC numbers. In order to explore this further, it was determined whether the decreased RBC number was caused by decreased red blood cell production, or increased red blood cell destruction. The spleens of HRI−/− mice were on average 10 fold larger at day 81 of low iron diet than +/+ mice both with and without diet-induced iron deficiency as shown by electron microscopic examination of the inclusions in the reticulocytes of HRI−/− mice in iron-deficiency. Blood from mice of all four groups at day 59 after receiving low iron diet, was collected and processed for EM. Inclusions were not observed in the blood samples of other groups. Tissue sections of the bone marrow and the spleen of iron deficient HRI−/− mice showed marked expansion of the red pulp by erythroid precursors in both the spleen and bone marrow. In addition, the reticulocyte count was elevated in HRI−/− mice both on a normal diet (8.6%) and on an iron deficient diet (11.5%) as compared to the HRI+/+ mice on either normal or iron deficient diet (2.3%).

The half-life of the RBC from both HRI +/+ and −/− mice on the normal diet and iron deficient diet was examined by in vivo labeling of RBC with biotin through tail vein injection as described (Jackson, R. J. (1991). Binding of Met-tRNA, H. Trachsel, ed. (Boca Raton: CRC Press); Baker, et al. (1997) Am. J. Hematol. 56, 17-25). There is no apparent difference in the survival of RBC from Wt and HRI−/− mice, either on normal or iron-deficient diet. The half-life of RBC is approximately 22 days, in good agreement with other published results (Levin, 1999). All these features indicate that the considerable decrease in red blood cell count was caused by abnormal destruction of red cell precursors with compensatory hyperplasia of the hematopoietic organs. Similar hematological changes were found in all animals examined continuously from 17 to 84 days of the low iron diet. No difference was observed in platelet counts. A general pathological survey of other tissues did not reveal significant morphological differences between −/− and +/+ mice placed on the low iron diet for 43-84 days. The hearts of HRI−/− mice were enlarged by about 50% in iron deficiency compared to Wt+Fe, Wt−Fe or Ko+Fe mice. This cardiomegaly is most likely a secondary response to the anemia, in light of the fact that HRI is not expressed in the heart.

These findings demonstrate that HRI−/− mice have dramatically altered the normal response to diet-induced iron deficiency and subsequent heme deficiency by shifting from an adaptive decrease in red blood cell volume and intracellular hemoglobin content to an increased production of abnormally dense red blood cells with compensatory hematopoietic hyperplasia.

Precipitation of Unbound Globins in Red Blood Cells of Iron-Deficient HRI−/− Mice

The molecular basis for the abnormal density of HRI−/− RBCs was then examined. Examination of Wright-Giemsa stained blood smears under the light microscope showed the presence of multiple variably sized eosinophilic inclusions within reticulocytes and, to a lesser extent, within fully mature RBCs in iron deficient HRI−/− mice. These inclusions were not discernable in RBC of +/+ mice. Upon staining of blood smears with crystal violet, granular bodies similar to Heinz bodies were seen in 80% of RBC from iron deficient HRI−/− mice. Heinz bodies were also seen in blood samples from HRI−/− mice on a normal diet, albeit to a lesser extent (9.6%). In contrast, RBC from +/+ mice on either normal or low iron diet did not contain Heinz bodies. Heinz bodies have been extensively characterized in RBCs of human patients with unstable hemoglobin syndromes, genetic defects in the hexose monophosphate shunt, thalassemias, and various chemical, particularly oxidative insults. In all cases, Heinz bodies are composed of denatured proteins, primarily globins (Beutler, E. (1983). Heinz body staining; Blood, marrow and iron staining.

In Hematology, W. J. Williams, Beutler, E., Erslev, A. J., and Lichman, M. A., ed.: McGraw-Hill Book company), pp. ppl603-1604). To further characterize the eosinophilic inclusion bodies found in the reticulocytes of HRI−/− mice, the blood cells were examined by transmission electron microscopy. The inclusion bodies were homogeneous in electron density and not bound by a membrane. Similar inclusions are present in patients with thalassemia (Polliack, et al. (1973) Brit. J. Haemat. 24, 319-326; Wickramasinghe, et al. (1984) Br J Haematol 56, 473-482; Wickramasinghe, et al. (1975) Brit. J.-Haemat. 30, 395-399; Wickramasinghe, et al. (1980) Brit. J. Haemat. 45, 401-404). In these cases, the inclusions have been shown to contain â or â′-globin in Alpha - or Alpha′-thalassemia respectively (Ho, et al. (1997) Blood 89, 322-328; Wickramasinghe, et al. (1996) Brit. J. Haemot. 93, 576-58). From the study of unstable hemoglobins caused by mutations that decrease heme incorporation (Dacie, et al. (1967) Nature 216,663-5; Wajcman, et al. (1992) Biochim Biophys Acta 1138,127-32) and from biochemical studies (Waks, et al. (1973) J. Biol. Chem. 248, 6462-6470; Yip, et al. (1972) J. Biol. Chem. 247, 7237-7244), it is well documented that globins misfold and precipitate in the absence of proper binding to heme. It is also well established that RBCs and late red blood cell precursors with inclusions are more susceptible to destruction. Therefore, the data are consistent with the hypothesis that, in the absence of HRI, globins lacking heme precipitate within RBC and their late precursors resulting in their destruction.

Increased Sensitivity of HRI−/− RBC to Hemolytic Reagents

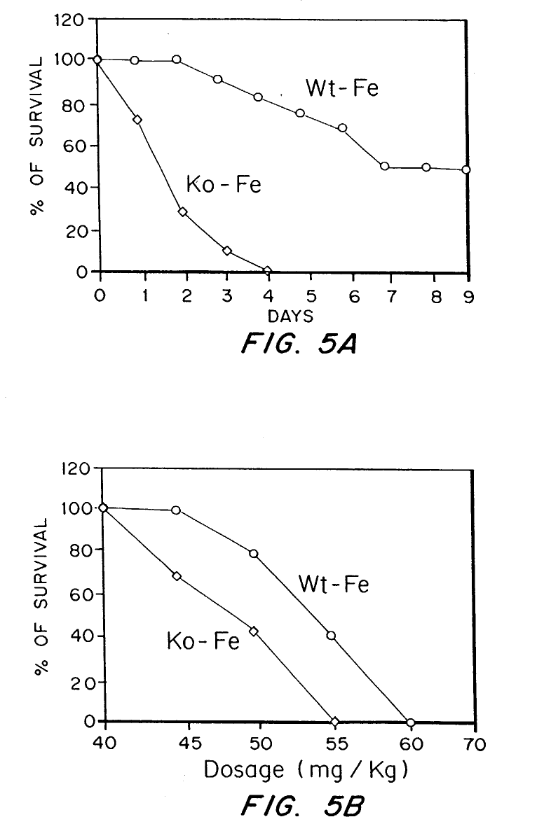

States of iron deficiency have been and remain very common in humans. Thus, exposure to situations that render RBCs more fragile may be often concurrent with iron-deficiency, such as in thalassemias, sickle cell disease, malaria and other infections, extreme climatic conditions, ingestion of natural toxins, heavy metals or drugs. Hence, it was investigated whether HRI−/− mice are more sensitive to hemolytic agents than normal mice. FIG. 5 shows the decreased survival of HRI−/− Mice in phenylhydrazine-induced hemolysis. Both Wt and HRI−/− mice were injected with phenylhydrazine at the dosages as indicated at Days 0, 1 and 3. The mice were observed daily. FIG. 5A shows the % of survival at different dosage at day 6 after initial phenylhydrazine injection. Six mice of each genotype were used for each dosage. FIG. 5B shows the time courses of survival of the iron deficient Wt and HRI−/− mice at the dosage of 50 mg/Kg (n=12 for wt mice; n=11 for HRI−/− mice). As shown in FIG. 5A, the survival rate of iron-deficient HRI−/− mice upon phenylhydrazine induced hemolytic erythroid stress is dramatically reduced. The standard phenylhydrazine regime employs three injections of phenylhydrazine on days 0, 1 and 3. After two injections, 90% of iron-deficient HRI−/− mice died, in contrast to nearly 100% survival of Wt mice with iron-deficiency. Even in the absence of iron-induced heme deficiency, HRI−/− mice were already more sensitive to phenylhydrazine with IC50 of 50 mg/Kg as compared to 55 mg/Kg of Wt mice (FIG. 5B). Thus, although HRI−/− RBCs have a normal half-life life on an iron-deficient diet, these RBCs are much more sensitive to additional stress such as oxidative stress induced by the administration of phenylhydrazine. This finding indicate that HRI provides a protective role in maintaining the integrity of mature red cells, particularly during combined assaults of iron-deficiency together with additional stress. Thus, even though HRI is no longer expressed in mature erythrocytes, its effect during erythroid differentiation persists into mature RBC.

Collectively, these findings strongly suggest that the increased cell destruction seen in HRI−/− mice on a low iron diet is caused by the excessive accumulation and precipitation of globin chains unbound by heme. The lack of HRI relieves the normal negative control on globin translation to be exerted in case of reduced heme concentration. Furthermore, in the absence of HRI, RBCs become much more sensitive to hemolytic agents, particularly in iron deficiency.

The results shown herein identify a clear physiological function of HRI by analyzing the phenotype of mice with a targeted disruption of the HRI gene. The generation of mice with a null HRI phenotype was evidenced by the absence of detectable HRI protein by Western blot analysis and the marked decrease in de novo eIF2 Alpha phosphorylation in the reticulocytes of HRI−/− homozygotes. It is demonstrated that HRI is essential to balance Alpha and Alpha′ globin synthesis with heme availability in erythroid cells. As expected from a protein expressed predominantly in erythroid cells, only RBCs and their precursors were found to be directly affected by the lack of HRI.

Our findings are summarized in the model shown in FIG. 5. Heme is essential for red blood cell development (reviewed in Sassa, S. (1988) Seminars in Hematology 25, 312-320; Fukuda et al., 1994; Nakajima, et al. (1999) EMBO Journal 18,6282-6289). One molecule of heme is incorporated into each of the Alpha and Alpha′ globin chains in the formation of a stable hemoglobin tetramer. HRI serves as a feedback inhibitor of globin synthesis by sensing heme. Heme binds to the kinase insertion domain of HRI and prevents its activation by autophosphorylation. Once HRI is activated, it phosphorylates eIF-2 and inhibits its recycling for another round of protein synthesis. Consistent with previous biochemical studies (reviewed in Chen, 2000), a 7 fold increase in protein synthesis, which also became insensitive to heme, was observed in the reticulocytes of HRI −/− mice with a corresponding decrease in eIF-2 Alpha phosphorylation

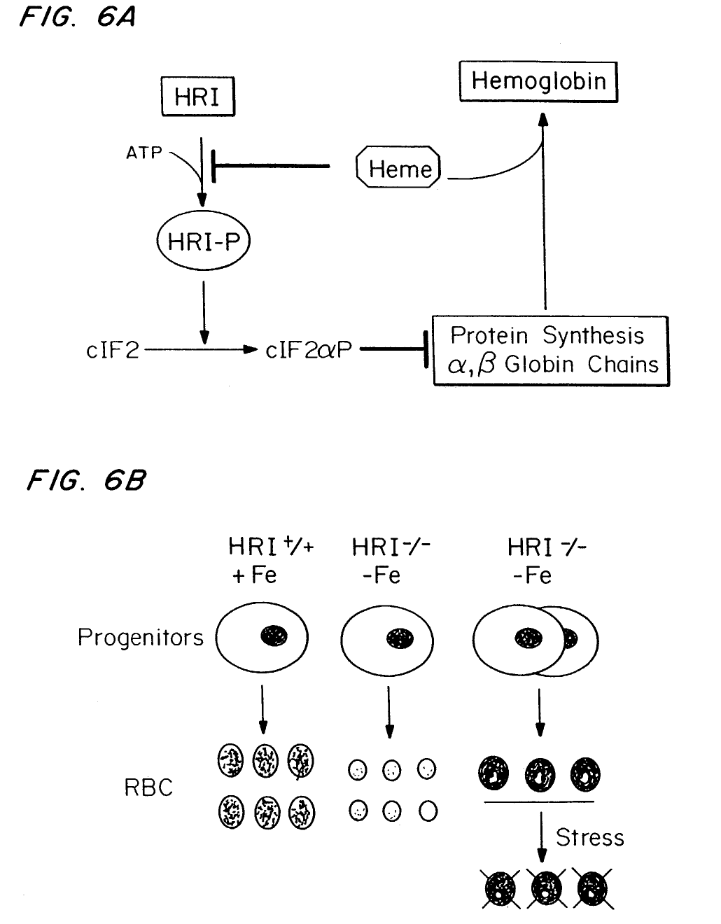

FIGS. 6A and 6B are schematics of models of the role of HRI duringerythroid differentiation. FIG. 6A shows regulation of alpha and beta globin synthesis by HRI and heme. FIG. 6B shows altered hematological response of HRI−/− mice to iron deficiency. As illustrated in FIG. 7B, the mild alterations of RBCs observed in HRI−/− mice in the absence of stress became profound under conditions of diet-induced iron deficiency, which decreases the intracellular concentration of heme. The normal hematological response of wild type mice to iron deficiency, characterized by a microcytic and hypochromic anemia, switched to a hyperchromic and hemolytic anemia with increased destruction of the late red cell precursors and compensatory erythroid hyperplasia. Destruction of mature red blood cells was exacerbated in the presence of hemolytic agents. Together, these data are consistent with the above model in which HRI normally insures that no globin chains are translated in excess of what can be assembled into hemoglobin tetramers for the amount of heme available. The critical role of HRI becomes, therefore, apparent only when heme concentrations in RBC precursors decline, as it is commonly found in iron deficiency.

The Lesser of Two Evils: Microcytic Anemia Rather than Hemolytic Anemia

Heme plays a key role in the general metabolism of every cell but is especially abundant in RBCs for the production of hemoglobin. One of the limiting steps in the biosynthesis of heme is the incorporation of iron into protoporphyrin by the enzyme ferrochelatase. Since the excess of free iron is very toxic for cells due to its oxidative properties, its intestinal absorption is tightly regulated, and tissular reserves in iron-ferritin complexes are limited (reviewed in Andrews, N. C. (2000) Nat. Rev. Genet. 1, 208-217). Iron deficiency is very common with an incidence of approximately 2 billion cases worldwide. It is most often a consequence of a low iron diet, or blood loss. When available iron and, as a consequence, heme declines below a certain threshold, the occurrence of anemia by decreased hemoglobin tetramer production is unavoidable. However, this data indicate that, in the absence of HRI, the consequences of iron deficiency are much more deleterious than with HRI. These findings are consistent with the hypothesis that the least detrimental adaptive response is to decrease globin production, resulting in mild microcytic and hypochromic anemia, rather than allowing globin translation to continue. If this occurs, as in the HRI−/− mice, free globins (unbound to heme) precipitate and add a major hemolytic component to the pathophysiology of the anemia. Microcytosis and hypochromia have been one of the first biological signs identified in human medicine and remain one of most frequent anomalies found in patients. Yet, no molecular mechanism had been identified for this phenomenon. The data indicate that HRI is responsible for this physiological adaptation of RBCs to iron deficiency. In addition, the occurrence of situations that render RBCs more fragile often coexists as in thalassemias, sickle cell disease, unstable hemoglobins, malaria and other infections, extreme climatic conditions, ingestion of natural toxins, heavy metals or drugs. In the absence of HRI, the combination of iron deficiency with one of these situations may be rapidly fatal.

Control of the Expression of Globin Genes by a General Translational Factor, eIF-2

Control of gene expression of single genes most often involves a form of regulation that targets a specific trans-acting factor acting on a gene specific cis-acting element in isolation or as part of a complex of factors. It might therefore be surprising that the control of the expression of alpha and beta globins by heme ultimately acts on the general translational factor eIF2. In this case, the specificity is achieved by the restriction of expression of the sensor/regulator HRI for the erythroid lineage, coupled to the fact that globin mRNAs are, by far, the main template for protein synthesis in reticulocytes. Since at least 25% of globin protein synthesis occurs in reticulocytes after nuclei have been extruded, translational regulation is the main level of control remaining (Beutler, E. (2001). Production and destruction of erythrocytes. In Williams Hematology, E. Beutler, et al, ed.: McGraw-Hill Book company), pp. 355-368).

Although the hyperplasia of hematopoietic organs may be a compensatory reaction to the accelerated red blood cell destruction in HRI−/− mice in iron deficiency, it may also be due to direct proliferative or anti-apoptotic effects triggered by the loss of HRI in RBC precursors.

Overexpression of dominant-negative mutants of HRI in murine erythroleukemia (MEL) cells by retrovirus-mediated gene transfer has been shown to increase the proliferative capacity of these cells upon induction of terminal differentiation by dimethylsulfoxide (Crosby, et al. (2000) Blood 96, 3241-3247).

eIF2 Alpha Kinases as Specific Sensors and Effectors That Protect Against Environmental Stress

In addition to HRI, three other eIF2a kinases, PKR, GCN2 and PERK, have been found in mammalian cells. The observation that the pathological consequences of the disruption of the mouse HRI gene are revealed under conditions of diet-induced iron deficiency is consistent with the fact that all eIF2Alpha kinases are activated under various stress conditions. Mice null for the ubiquitous kinase PKR are also viable without significant phenotypic change until challenged by viral infection [Yang, 1995 #1320; (Abraham, et al. (1999) PKR. J Biol Chem 274, 5953-5962; Stojdl, et al. (2000) J Virol 74, 9580-9585). In yeast, GCN2 is non-essential under optimal growth conditions (Hinnebusch, 1996). Thus, the physiological role of members of this class of kinases may be to act as homeostatic guardians against major environmental stress by ultimately regulating protein synthesis in response to specific exogenous signals.

These findings provide important insights into the molecular mechanisms by which HRI coordinates the synthesis of globins in red blood cell precursors to the concentration of heme in vivo.

Modifications and variations of the present invention will be obvious to those skilled in the art from the foregoing detailed description. Such modifications and variations are intended to come within the scope of the following claims.

I claim:

1. An animal model for screening for the role of HRI, or for compounds affecting HRI activity, containing cells comprising one or more inactive genes encoding HRI, or containing an inactive regulatory sequence thereof.

2. The animal model of claim 1, wherein the animal is heterozygous for HRI-.

3. The animal model of claim 1, wherein the animal is homozygous for HRI-.

4. The animal model of claim 1, wherein the animal is a mouse.

5. A method for identifying compounds altering HRI activity comprising:
(a) screening for the role of HRI, or for compounds affecting HRI activity, in an animal model containing cells comprising one or more inactive genes encoding HRI, or containing an inactive regulatory sequence thereof,
(b) exposing the animal to the compound to be screened, and
(c) determining an effect in the animal model.

6. The method of claim 5, wherein the HRI activity is induced, repressed, or attenuated.

7. The method of claim 6, wherein the HRI activity is measured by gene expression, protein synthesis, eIF-2 phosphorylation or kinase activity, or cellular phenotype.

8. The method of claim 5, wherein the animal is a mouse.

9. The method of claim 6, wherein the mouse is heterozygous for HRI-.

10. The method of claim 6, wherein the mouse is homozygous for HRI-.

11. The method of claim 5 wherein the compounds are useful for the treatment of conditions selected from the group consisting of psoriasis, chronic myelogenous leukemia, cancer, and a cell proliferation disorder.

12. A compound for altering HRI activity identified by the method of claim 5.

13. A method of treating a disorder wherein RBCs are more fragile comprising administering HRI, or a compound inducing HRI inducing expression or activity, or mimicking HRI activity to the patient in need thereof,
wherein the disorder is selected from the group consisting of thalassemias, sickle cell disease, unstable hemoglobins, malaria and other infections, extreme climatic conditions, and ingestion of natural toxins, heavy metals or drugs.

14. The method of claim 13 wherein the HRI is administered as a protein in a pharmaceutical composition.

15. The method of claim 13, wherein the HRI is encoded in a nucleic acid.

16. The method of claim 15, wherein the nucleic acid is encoded in a viral vector.

17. The method of claim 16, wherein the viral vector is a retroviral vector.
